(19) United States
(12) Patent Application Publication
(10) Pub. No.: US 2024/0299322 A1
(43) Pub. Date: Sep. 12, 2024

(54) TREATING CANCERS WITH COMBINATIONS OF PARP INHIBITOR AND ACYLFULVENES

(71) Applicant: LANTERN PHARMA INC., Dallas, TX (US)

(72) Inventors: Aditya Kulkarni, Dallas, TX (US); Jianli Zhou, Dallas, TX (US); Kishor Bhatia, Ellicott, MD (US)

(73) Assignee: LANTERN PHARMA INC., Dallas, TX (US)

(21) Appl. No.: 18/424,930

(22) Filed: Jan. 29, 2024

Related U.S. Application Data
(63) Continuation of application No. PCT/US22/74314, filed on Jul. 29, 2022.
(60) Provisional application No. 63/365,995, filed on Jun. 7, 2022.

(51) Int. Cl. A61K 31/17; A61K 31/122; A61K 31/337; A61K 31/502; A61K 31/5025; A61K 33/243; A61P 35/00

(52) U.S. Cl. CPC A61K 31/17; A61K 31/122; A61K 31/337; A61K 31/502; A61K 31/5025; A61K 33/243; A61P 35/00

(57) ABSTRACT

A method of treating cancer includes a combination of a therapeutically effective amount of an illudin or an illudin analog thereof, derivative, or a pharmaceutically acceptable salt thereof, and a therapeutically effective amount of a PARP inhibitor or an analog, derivative, or a pharmaceutically acceptable salt thereof. Compositions and kits of the same are included herein.

### CROSS-REFERENCE TO RELATED APPLICATION

This application is a continuation of International Patent Application No. PCT/US22/74314, filed on Jul. 29, 2022, which claims the benefit of U.S. Provisional Patent Application No. 63/227,260, filed Jul. 29, 2021 and U.S. Provisional Patent Application No. 63/365,995, filed Jul. 6, 2022, each of which is incorporated by reference herein in its entirety.

### TECHNICAL FIELD

This application relates to cancer treatments and more specifically this application relates to cancer treatments using a combination therapy including a PARP inhibitor.

### BACKGROUND

Cancer is one of the most common causes of death in people. The development of therapeutic strategies for patients with advanced cancer has markedly improved overall survival. However, resistance to anticancer reagents is inevitable, and the prognosis of advanced cancer remains poor. There are several potential sources of cancer drug resistance, including alterations to drug transporters, the suppression of apoptosis, mitochondrial alterations, the promotion of DNA damage repair, autophagy, epithelial-mesenchymal transition, and cancer stem cells (CSCs). Appropriate strategies that consider the mechanisms are necessary to cure cancer.

Combination-therapy treatments for cancer have become more common, in part due to the perceived advantage of attacking the disease via multiple avenues. Although many effective combination-therapy treatments have been identified over the past few decades; in view of the continuing high number of deaths each year resulting from cancer, a continuing need exists to identify effective therapeutic regimens for use in anticancer treatment. Accordingly, there is always a need for improved methods to treat cancer.

### SUMMARY

This application discloses the discovery that treatment of a cancer in a subject with a combination of an illudin or illudin analog (e.g., acylfulvene) and a PARP inhibitor has synergistically greater effects (i.e., greater than the effects of each added together) than the effects provided by either acylfulvene or PARP inhibitor treatment, alone. For example, the cytotoxicity delivered from treating a cancer with a combination of illudin or acylfulvene and a PARP inhibitor is unexpectedly greater compared to the cytotoxicity delivered when treating the cancer with illudin or acylfulvene or PARP inhibitor, alone (or greater than the cytotoxicity of both added together). In addition, it was discovered that the combination therapy results in more rapid killing of cancer cells and more rapid tumor shrinkage than was found when either therapy, alone, was used.

One aspect of this application includes a combination therapy for treating cancers. In embodiments, the therapy includes administering a combination of active agents including an illudin or illudin analog (e.g., acylfulvene), and a PARP inhibitor.

Another aspect of this application provides pharmaceutical compositions comprising an illudin or illudin analog (e.g., acylfulvene) and a PARP inhibitor or pharmaceutically acceptable salts thereof, mixed with pharmaceutically suitable carriers or excipient(s) at doses to treat or prevent cancer. The pharmaceutical compositions can also be administered in combination with other therapeutic agents or therapeutic modalities simultaneously, sequentially, or in alternation.

Another aspect of this application includes therapeutically effective amounts of each illudin or acylfulvene, and a PARP inhibitor used in combination that will be lower when used in combination in comparison to monotherapy with each agent alone. Such lower therapeutically effective amounts could afford for lower toxicity of the therapeutic regimen.

Another aspect of this application includes the therapy including an acylfulvene that is (+)-hydroxyureamethyl acylfulvene.

Another aspect of this application includes the therapy including an acylfulvene that is Irofulven.

Another aspect of this application includes the treatments of cancer that can include solid tumors, and hematological malignancies. Tumors such as, but not limited to, hyperplastic or neoplastic disease, such as a carcinoma, sarcoma, or mixed type cancer, including breast, colon, rectal, endometrial, gastric, prostate or brain, mesothelioma, ovarian, lung or pancreatic cancer can be targeted for therapy.

### BRIEF DESCRIPTION OF THE FIGURES

FIG. 1 shows the effect of the combination of LP-184 with Olaparib on the survival of cells;

FIG. 2 shows the effect of the combination of LP-100 with either Olaparib or Rucaparib on shrinking tumors in prostate xenograft mouse models in vivo;

FIG. 3A shows IC50 data in which an Olaparib single treatment was 1248.1 nM;

FIG. 3B shows IC50 data in which an Olaparib single treatment was 108.2 nM respectively;

FIG. 3C shows IC50 data in which Olaparib and LP-184 co-treatment was 278.6 nM Olaparib and 13.93 nM LP-184;

FIG. 3D shows the synergistic effect of LP-184 and Olaparib analyzed through in Isobole plot;

FIG. 4A shows IC50 data in which a Talazoparib single treatment was 13.1 nM;

FIG. 4B shows IC50 data in which a Talazoparib single treatment was 87.4 nM;

FIG. 4C shows IC50 data in which a Talazoparib and LP-184 co-treatment was 3.6 nM Olaparib and 7.2 nM LP-184;

FIG. 4D shows the synergistic effect of LP-184 and Talazoparib analyzed through an Isobole plot.

### DETAILED DESCRIPTION

This application provides a combination therapy for treating solid cancers and blood cancers. In embodiments, the therapy includes administering a combination of active agents including an illudin or an illudin analog (e.g., acylfulvene) and a PARP inhibitor. In other embodiments, the therapy includes administering a combination of other therapies. In other embodiments, the combination therapy can be used to treat biochemical occurrence or recurrence of solid cancers (e.g., lung cancer, breast cancer, ovarian cancer, prostate cancer, colon cancer, rectum cancer, and bladder cancer), glioblastoma and atypical teratoid rhabdoid, and renal cell carcinoma). In other embodiments, the therapy includes the combination therapy that can be used to treat biochemical occurrence and recurrence of blood cancers in which an acylfulvene (e.g., hydroxyureamethyl acylfulvene) or salt thereof and a PARP inhibitor administered in a therapeutically effective amount to the patient. In certain embodiments, the combination can provide a treatment for lymphoma, such as mantle cell lymphoma (MCL) and double-hit lymphoma (DHL). In multiple myeloma (MM), the overgrowth of plasma cells in the bone marrow can crowd out normal blood-forming cells.

### Illudin or Acylfulvene

In one embodiment, this application includes the use of an illudin or illudin analog (e.g., acylfulvene). Acylfulvene is a class of cytotoxic semi-synthetic derivatives of illudin, a natural product that can be extracted from the jack o′lantern mushroom (Omphalotus olearius). Acylfulvene, derived from the sesquiterpene illudin S by treatment with acid (reverse Prins reaction), is far less reactive to thiols than illudin S.

In one example, the acylfulvene is (−)-hydroxyureamethyl acylfulvene (termed LP-184 by Lantern Pharma Inc.), which shifts light positively, is shown below:

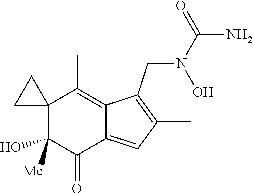 [SMILES: CC1=C(CN(O)C(N)=O)C2=C(C)C3(CC3)[C@@](C)(O)C(=O)C2=C1]

In another example, the acylfulvene is (+)-hydroxyureamethyl acylfulvene (termed LP-284 by Lantern Pharma Inc.), which shifts light negatively, is shown below:

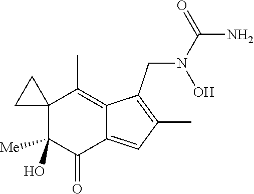 [SMILES: CC1=C(CN(O)C(N)=O)C2=C(C)C3(CC3)[C@](C)(O)C(=O)C2=C1]

(+)-hydroxyureamethyl acylfulvene and (−)-hydroxyureamethyl acylfulvene are enantiomers and are now known publicly.

In another example, the acylfulvene is Irofulven.

### PARP Inhibitor

PARP inhibitor are a type of cancer drug. PARP stands for poly adenosine diphosphate-ribose polymerase (poly-ADP ribose polymerase (PARP) inhibitor), a type of enzyme that helps repair DNA damage in cells. PARP inhibitor work by preventing cancer cells from repairing damaged DNA, allowing them to die. PARP enzymes help repair DNA damage. Blocking them can keep cancer cells from repairing, and this allows them to die. There are at least four main PARP inhibitor olaparib (Lynparza), niraparib (Zejula), rucaparib (Rubraca) and talazoparib (Talzenna). Pharmaceutical compositions are disclosed that include one or more PARP inhibitor and typically at least one additional substance, such as an excipient, a known therapeutic other than those of the present disclosure, and combinations thereof. In some embodiments, a PARP inhibitor can be used in combination with other agents known to have beneficial, additive or synergistic activity with the PARP inhibitor. In one specific embodiment, the PARP inhibitor is a PARP-1 inhibitor. In other embodiment, the PARP inhibitor is an inhibitor of any enzyme of the PARP family, e.g., PARP1 and/or PARP2.

Examples of suitable PARP inhibitors according to the invention include, but are not limited to, olaparib (AZD-2281, 4-[(3-[(4-cyclopropylcarbonyl)piperazin-4-yl]carbonyl)-4-fluorophenyl]met-hyl(2H)-phthalazin-1-one), veliparib (ABT-888, CAS 912444-00-9, 2-((fi)-2-methylpyrrolidin-2-yl)-1W-benzimidazole-4-carboxamide), CEP-8983 (ll-methoxy-4,5,6,7-tetrahydro-1H-cyclopenta[a]pyrrolo[3,4-c]car-bazole-1,3(2H)-dione) or a prodrug thereof (e.g. CEP-9722), rucaparib (AG014699, PF-01367338, 8-Fluoro-2-{4-[(methylamino)methyl]phenyl}-1,3,4,5-tetrahydro-6H-azepino[-5,4,3-cd]indol-6-one), E7016 (GPI-21016, 10-((4-Hydroxypiperidin-1-yl)methyl)chromeno-[4,3,2-de]phthalazin-3(2H)-o-ne), talazoparib (BMN-673, (8S,9R)-5-fluoro-8-(4-fluorophenyl)-9-(1-methyl-1H-1,2,4-triazol-5-yl)-8,-9-dihydro-2H-pyrido[4,3,2de]phthalazin-3 (7H)-one), INO-1001 (4-phenoxy-3-pyrrolidin-1-yl-5-sulfamoyl-benzoic acid), KU0058684 (CAS 623578-11-0), niraparib (MK 4827, Merck & Co Inc), iniparib (BSI 201), iniparib-met (C-nitroso metabolite of Iniparib), CEP 9722 (Cephalon Inc), LT-673, MP-124, NMS-P118, XAV939, AZD 2461, nicotinamides, 5-methyl nicotinamide, 4-Amino-1,8-naphthalimide, picolinamide, benzamides, 3-substituted benzamides, 3-methoxybenzamide, 3-hydroxybenzamide, 3-aminobenzamide, 3-chloroprocainamide, 3-nitrosobenzamide, 4-aminobenzamide, 2-aminobenzamide, methyl 3,5-diiodo-4-(4′-methoxyphenoxy) benzoate, methyl-3,5-diiodo-4-(4′-methoxy-3′,5′-diiodo-phenoxy) benzoate, cyclic benzamides, 1,5-di[(3-carbamoylphenyl)aminocarbonyloxy] pentane, indoles, benzimidazoles, benzoxazole-4-carboxamides, benzimidazole-4-carboxamides, 2-substituted benzoxazole 4-carboxamides, 2-substituted benzimidazole 4-carboxamides, 2-aryl benzimidazole 4-carboxamides, 2-cycloalkylbenzimidazole-4-carboxamides, 2-(4-hydroxphenyl) benzimidazole A-carboxamide, quinoxalinecarboxamides, imidazopyridinecarboxamides, 2-phenylindoles, 2-substituted benzoxazoles, 2-phenyl benzoxazole, 2-(3-methoxyphenyl) benzoxazole, 2-substituted benzimidazoles, 2-phenyl benzimidazole, 2-(3-methoxyphenyl) benzimidazole, 1,3,4,5-tetrahydro-azepino[5,4,3-cd]indol-6-one, azepinoindoles, azepinoindolones, 1,5-dihydro-azepino[4,5,6-cd]indolin-6-one, dihydrodiazapinoindolinone, 3-substituted dihydrodiazapinoindolinones, 3-(4-trifluoromethylphenyl)-dihydrodiazapinoindolinone, tetrahydrodiazapinoindolinone, 5,6-dihydroimidazo[4,5,1-j,k][1,4]benzodiazopin-7(4H)-one, 2-phenyl-5,6-dihydro-imidazo[4,5,1-jk][1,4]benzodiazepin-7(4H)-one, 2,3-dihydro-isoindol-1-one, benzimidazole-2-piperazine, benzimidazole-2-piperazine heterocyclic derivatives, 4-iodo-3-nitrobenzamide, benzopyrones, 1,2-benzopyrone 6-nitrosobenzopyrone, 6-nitroso 1,2-benzopyrone, 5-iodo-6-aminobenzopyrone, benzoylurea, quinolone, isoquinolone, isoquinolinones, dihydroisoquinolinones, 2H-isoquinolin-1-ones, 3H-quinazolin-4-ones, 5-substituted dihydroisoquinolinones, 5-hydroxy dihydroisoquinolinone, 5-methyl dihydroisoquinolinone, 5-hydroxy isoquinolinone, 5-amino isoquinolin-1-one, 5-dihydroxyisoquinolinone, 1,5-dihydroxyisoquinoline, 1,5-isoquinolinediol, 4-hydroxyquinazoline, substituted thiazolyl-isoquinolinones, substituted oxazoyl-isoquinolinones, tetrahydro-2H-isoquinolin-1-one, 3,4-dihydroisoquinolin-1(2H)-ones, 3,4-dihydro-5-methoxy-isoquinolin-1(2H)-one, 3,4-dihydro-5-methyl-1(2H)isoquinolinone, 3H-quinazolin-4-one, isoquinolin-1(2H)-ones, 3,4 dihydroisoquinolin-1(2H)-one, 4-carboxamido-benzimidazole, substituted 6-cyclohexylalkyl substituted 2-quinolinones, substituted 6-cyclohexylalkyl substituted 2-quinoxalinones, 7-phenylalkyl substituted 2-quinolinones, 7-phenylalkyl substituted 2-quinoxalinones, 6-substituted 2-quinolinones, 6-substituted 2-quinoxalinones, 1-(arylmethyl)quinazoline-2,4(1H,3H)-dione, 4,5-dihydro-imidazo[4,5,1-ij]quinolin-6-ones, 1,6-naphthyridine-5(6H)-ones, 1,8-naphthalimides, 4-amino-1,8-naphthalimides, 3,4-dihydro-5-[4-1(1-piperidinyl)butoxy]-1(2H)-isoquinolinone, 2,3-dihydrobenzo[de]isoquinolin-1-one, 1-1 lb-dihydro-[2H]benzopyrano[4,3,2-de]isoquinolin-3-one, tetracyclic lactams, benzpyranoisoquinolinones, benzopyrano[4,3,2-de]isoquinolinone, quinazolines, quinazolinones, quinazolinediones, A-hydroxyquinazoline, 2-substituted quinazolines, 8-hydroxy-2-methylquinazolin-4-(3H)one, phthalazines, phthalazinones, phthalazin-1(2H)-ones, 5-methoxy-4-methyl-1(2) phthalazinones, 4-substituted phthalazinones, 4-(1-piperazinyl)-1(2H)-phthalazinone, tetracyclic benzopyrano[4,3,2-de]phthalazinones and tetracyclic indeno[1,2,3-de]phthalazinones, tricyclic phthalazinones, 2-aminophthalhydrazide, phthalazinone ketone, dihydropyridophthalazinone, 6-substituted 5-arylamino-1 h-pyridine-2-ones, pyridazinones, tetrahydropyridopyridazinone, tetraaza phenalen-3-one, thieno[2,3-c]isoquinolin-5-one (TIQ-A), 2,5-diazabicyclo[2.2.1]heptane, pyrimidoimidazole, isoindolinones, phenanthridines, phenanthridinones, 5 [H]phenanthridin-6-one, substituted 5[H] phenanthridin-6-ones, 2,3-substituted 5 [H]phenanthridin-6-ones, sulfonamide/carbamide derivatives of 6(5H)phenanthridinones, thieno[2, 3-c]isoquinolones, 9-amino thieno[2,3-c]isoquinolone, 9-hydroxythieno[2,3-c]isoquinolone, 9-methoxythieno[2,3-c]isoquinolone, N-(6-oxo-5,6-dihydrophenanthridin-2-yl]-2-(N,N-dimethylamino}acetamide, substituted 4,9-dihydrocyclopenta[imn]phenanthridine-5-ones, unsaturated hydroximic acid derivatives, O-(3-piperidino-2-hydroxy-1-propyl)nicotinic amidoxime, O-(2-hydroxy-3-piperidino-propyl)-3-carboxylic acid amidoxime, pyridazines, pyrazinamide, BGB-290, PF-1367338 (Pfizer Inc), AG014699 (Pfizer, Inc.), KU-59436 (KuDOS/AstraZeneca PJ34, 4-amino-1,8-naphthalimide (Trevigen), 6(5H)-phenanthridinone (Trevigen), NU1025, 4-HQN, BGP-15, A-966492, GPI21016, 6(5H)-phenanthridinone (Phen), theobromine, theophylline, caffeine, methylxanthines, thymidine, 3-aminophtalhydrazide, analogs, derivatives or a mixture thereof.

Additional PARP inhibitors are described for example in WO14201972, WO14201972, WO12141990, WO10091140, WO9524379, WO09155402, WO009046205, WO08146035, WO08015429, WO0191796, WO0042040, US2006004028, EP2604610, EP1802578, CN104140426, CN104003979, U.S. Pat. Nos. 6,022,935, 7,041,675, WO07041357, WO2003057699, U.S. Ser. No. 06/444,676, US20060229289, US20060063926, WO2006033006, WO2006033007, WO03051879, WO2004108723, WO2006066172, WO2006078503, US20070032489, WO2005023246, WO2005097750, WO2005123687, WO2005097750, U.S. Pat. Nos. 7,087,637, 6,903,101, WO20070011962, US20070015814, WO2006135873, UA20070072912, WO2006065392, WO2005012305, WO2005012305, EP412848, EP453210, EP454831, EP879820, EP879820, WO030805, WO03007959, U.S. Pat. No. 6,989,388, US20060094746, EP1212328, WO2006078711, U.S. Ser. No. 06/426,415, U.S. Ser. No. 06/514,983, EP1212328, US20040254372, US20050148575, US20060003987, U.S. Ser. No. 06/635,642, WO200116137, WO2004105700, WO03057145A2, WO2006078711, WO2002044157, US20056924284, WO2005112935, US20046828319, WO2005054201, WO2005054209, WO2005054210, WO2005058843, WO2006003146, WO2006003147, WO2006003148, WO2006003150, WO2006003146, WO2006003147, UA20070072842, U.S. Ser. No. 05/587,384, US20060094743, WO2002094790, WO2004048339, EP1582520, US20060004028, WO2005108400, U.S. Pat. No. 6,964,960, WO20050080096, WO2006137510, UA20070072841, WO2004087713, WO2006046035, WO2006008119, WO06008118, WO2006042638, US20060229289, US20060229351, WO2005023800, WO1991007404, WO2000042025, WO2004096779, U.S. Pat. No. 6,426,415, WO2068407, U.S. Pat. No. 6,476,048, WO2001090077, WO2001085687, WO2001085686, WO2001079184, WO2001057038, WO2001023390, WO01021615A1, WO2001016136, WO2001012199, WO95024379, WO200236576, WO2004080976, WO2007149451, WO2006110816, WO2007113596, WO2007138351, WO2007144652, WO2007144639, WO2007138351, WO2007144637, Banasik et al. (J. Biol. Chem., 267:3, 1569-75, 1992), Banasik et al. (Molec. Cell. Biochem, 138:185-97, 1994), Cosi et al. (Expert Opin. Ther. Patents 12 (7), 2002), Southan and Szabo (Curr Med Chem, 10 321-340, 2003), Underhill C. et al. (Annals of Oncology, doi:10.1093/annonc/mdq322, pp 1-12, 2010), Murai J. et al. (J. Pharmacol. Exp. Ther., 349:408-416, 2014), all these patents and publications being hereby incorporated by reference in their entirety.

In a preferred embodiment, the PARP inhibitor compound is selected from the group consisting of rucaparib (AG014699, PF-01367338), olaparib (AZD2281), veliparib (ABT888), iniparib (BSI 201), niraparib (MK 4827), talazoparib (BMN673), AZD 2461, CEP 9722, E7016, INO-1001, LT-673, MP-124, NMS-P118, XAV939, analogs, derivatives or a mixture thereof.

In an even more preferred embodiment, the PARP inhibitor is selected from the group consisting of rucaparib (AG014699, PF-01367338), olaparib (AZD2281), veliparib (ABT888), iniparib (BSI 201), niraparib (MK 4827), talazoparib (BMN673), AZD 2461, analogs, derivatives or a mixture thereof.

In one embodiment, acylfulvene or hydroxyureamethyl acylfulvene or its salt may be administered either prior to, concomitantly with, or subsequent to the administration of a PARP inhibitor.

One aspect of this application includes a method of treating cancer in a subject in need thereof. The method involves administering to the subject an effective amount of PARP inhibitor and an effective amount of an acylfulvene. PARP inhibitor may be administered prior to or concomitantly with an acylfulvene for optimal synergistic effects.

Another embodiment includes a pharmaceutical composition having a therapeutically effective amount of an illudin or an illudin analog thereof, derivative, or a pharmaceutically acceptable salt thereof; and a therapeutically effective amount of a PARP inhibitor or an analog, derivative, or a pharmaceutically acceptable salt thereof. The illudin analog can be HydroxyUreaMethylAcylfulvene.

In another embodiment, a kit for the treatment of cancer in a subject includes a therapeutically effective amount of an illudin or an illudin analog thereof, derivative, or a pharmaceutically acceptable salt thereof; and a therapeutically effective amount of a PARP inhibitor or an analog, derivative, or a pharmaceutically acceptable salt thereof:

In another embodiment, the second therapeutic is one or more chemotherapeutic agents selected from camptothecin derivatives, paclitaxel, docetaxel, epothilone B, 5-FU, gemcitabine, oxaliplatin, cisplatinum, carboplatin, melphalam, dacarbazine, temozolomide, doxorubicin, imatinib, erlotinib, bevacizumab, cetuximab and a Raf kinase inhibitor.

In another embodiment, the second therapeutic is one or more chemotherapeutic agents selected from paclitaxel or cisplatinum.

The term “combination therapy” can include or includes the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In another aspect, a composition or combination therapy herein, or a pharmaceutically acceptable salt or solvate thereof, may be administered in combination with radiation therapy. Radiation therapy can also be administered in combination with a composition of the present invention and another chemotherapeutic agent described herein as part of a multiple agent therapy.

Combination therapy can be achieved by administering two or more agents, e.g., an acylfulvene, a PARP inhibitor and one or more other therapeutic agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present within the patient's body at the same time, this need not be so.

The term “PARP inhibition” indicates a decrease in the baseline activity of a biological activity or process, namely, the activity of PARP. PARP inhibition relies mainly on two different mechanisms: (i) catalytic inhibition that act mainly by inhibiting PARP enzyme activity and (ii) bound inhibition that block PARP enzyme activity and prevent its release from the damage site. “Inhibition of PARP” refers to a decrease in the activity of PARP as a direct or indirect response to the presence of at least one compound and/or at least one pharmaceutically acceptable salt disclosed herein, relative to the activity of PARP in the absence of the at least one compound and/or the at least one pharmaceutically acceptable salt thereof. The decrease in activity can be due to the direct interaction of the at least one compound, stereoisomers thereof, and pharmaceutically acceptable salts thereof disclosed herein with PARP, or due to the interaction of the at least one compound and/or at least one pharmaceutically acceptable salt disclosed herein, with one or more other factors that in turn affect PARP activity. For example, the presence of at least one compound, stereoisomers thereof, and pharmaceutically acceptable salts thereof disclosed herein, may decrease PARP activity by directly binding to the PARP, by causing (directly or indirectly) another factor to decrease PARP activity, or by (directly or indirectly) decreasing the amount of PARP present in the cell or organism.

The methods of combination therapy may or should result in a synergistic effect, wherein the effect of a combination of compounds or other therapeutic agents is greater than the sum of the effects resulting from administration of any of the compounds or other therapeutic agents as single agents. A synergistic effect may also be an effect that cannot be achieved by administration of any of the compounds or other therapeutic agents as single agents. The synergistic effect may include, but is not limited to, an effect of treating cancer by reducing tumor size, inhibiting tumor growth, or increasing survival of the subject. The synergistic effect may also include reducing cancer cell viability, inducing cancer cell death, and inhibiting or delaying cancer cell growth.

Therapeutically effective doses can vary, as recognized by those skilled in the art, depending on the diseases treated, the severity of the disease, the route of administration, the age and general health condition of the patient, excipient usage, the possibility of co-usage with other therapeutic treatments such as use of other agents and the judgment of the treating physician. For example, guidance for selecting an effective dose can be determined by reference to the prescribing information for acylfulvene or hydroxyureamethyl acylfulvene or journal discussion the same.

The term “effective amount” as used herein refers to the amount of an agent needed to alleviate at least one or more symptoms of the disease or disorder, and relates to a sufficient amount of pharmacological composition to provide the desired effect. The term “therapeutically effective amount” therefore refers to an amount of the agent that is sufficient to provide a particular effect when administered to a typical subject. An effective amount may be an amount sufficient to decrease the symptoms of a disease responsive to inhibition of PARP. For cancer therapy, efficacy in vivo can, for example, be measured by assessing the duration of survival, time to disease progression (TTP), the response rates (RR), duration of response, and/or quality of life. Effective amounts may vary, as recognized by those skilled in the art, depending on route of administration, excipient usage, and co-usage with other agents. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

The dosage ranges for the administration of an agent according to the methods described herein depend upon, for example, the form of the agent, its potency, and the extent to which symptoms, markers, or indicators of a condition described herein are desired to be reduced, for example, the percentage reduction desired for tumor growth. The dosage should not be so large as to cause adverse side effects. Generally, the dosage will vary with the age, condition, and sex of the patient and can be determined by one of skill in the art. The dosage can also be adjusted by the individual physician in the event of any complication.

The term “therapeutically effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer. In another aspect, the disease or condition to be treated is a cell proliferative disorder.

The efficacy of an agent described herein in, e.g., the treatment of a condition described herein, or to induce a response as described herein (e.g., solid cancers or blood cancers) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. tumor size and/or growth rate. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example, treatment of blood cancers in a mouse model. When using an experimental animal model, efficacy of treatment is evidenced when a statistically significant change in a marker is observed, e.g. tumor size and/or growth rate. In some embodiments, the therapeutically effective amount of hydroxyureamethyl-acylfulvene, acylfulvene, Irofulven or a pharmaceutically acceptable salt thereof is selected from the group consisting of 0.5 mg/day, 1 mg/day, 2.5 mg/day, 5 mg/day, 10 mg/day, 20 mg/day, 30 mg/day, 60 mg/day, 90 mg/day, 120 mg/day, 150 mg/day, 180 mg/day, 210 mg/day, 240 mg/day, 270 mg/day, 300 mg/day, 360 mg/day, 400 mg/day, 440 mg/day, 480 mg/day, 520 mg/day 580 mg/day, 600 mg/day, 620 mg/day, 640 mg/day, 680 mg/day, and 720 mg/day.

The administration dose should be adjusted for the requirement of the individual in need For example, the administered dosage of the PARP inhibitor is 1-120 mg (in terms of the parent compound), preferably, 1-80 mg (in terms of the parent compound), and the administration frequency is twice a day (BID); the administered dosage of the PARP inhibitor is 1-120-240 mg (in terms of the parent compound), preferably, 60-120 mg (in terms of the parent compound), and the administration frequency is once a day (QD). In some cases, it is more suitable to apply the lower end of the above described dosage ranges, while in other cases the higher dosages may be used without causing harmful side effects.

The term “treat” is used and includes both therapeutic treatment and prophylactic treatment (reducing the likelihood of development). Both terms mean decrease, suppress, attenuate, diminish, arrest, or stabilize the development or progression of a disease (e.g., a disease or disorder delineated herein), lessen the severity of the disease or improve the symptoms associated with the disease.

The pharmaceutical compositions can be included in a container, pack, or dispenser together with instructions for administration.

The composition of the present invention is capable of further forming salts. The composition of the present invention can form more than one salt per molecule, e.g., mono-, di-, tri-. All of these forms are also contemplated within the scope of the claimed invention.

As used herein, “pharmaceutically acceptable salts” refer to derivatives of the compounds of the present invention wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present invention also encompasses salts formed when an acidic proton in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates), of the same salt.

As used herein, the term “selectively” means tending to occur at a higher frequency in one population than in another population. The compared populations can be cell populations. Preferably, an event occurs selectively in population A relative to population B if it occurs greater than two times more frequently in population A as compared to population B. An event occurs selectively if it occurs greater than five times more frequently in population A. An event occurs selectively if it occurs greater than ten times more frequently in population A; more preferably, greater than fifty times; even more preferably, greater than 100 times; and most preferably, greater than 1000 times more frequently in population A as compared to population B. For example, cell death would be said to occur selectively in cancer cells if it occurred greater than twice as frequently in cancer cells as compared to normal cells.

The composition, or pharmaceutically acceptable salts or solvates thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the invention can be found in Remington: the Science and Practice of Pharmacy, 19.sup.th edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

As used herein, a “subject in need thereof” is a subject having a precancerous condition. Preferably, a subject in need thereof has cancer. A “subject” includes a mammal. The mammal can be e.g., any mammal, e.g., a human, primate, bird, mouse, rat, dog, cat, cow, horse, goat, camel, sheep or a pig. Preferably, the mammal is a human. The subject of the present invention includes any human subject who has been diagnosed with, has symptoms of, or is at risk of developing a cancer or a precancerous condition.

A subject in need thereof may have refractory or resistant cancer. “Refractory or resistant cancer” means cancer that does not respond to treatment. The cancer may be resistant at the beginning of treatment or it may become resistant during treatment. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy. In certain embodiments the prior therapy is monotherapy. In certain embodiments the prior therapy is combination therapy.

In some embodiments, a subject in need thereof may have a secondary cancer as a result of a previous therapy. “Secondary cancer” means cancer that arises due to or as a result from previous carcinogenic therapies, such as chemotherapy.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer results in a decrease in number and size of tumors. Preferably, after treatment, tumor number or size is reduced by 5% or greater relative to number prior to treatment; more preferably, tumor number or size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present invention, or a pharmaceutically acceptable salt or solvate thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present invention, or a pharmaceutically acceptable salt or solvate thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating or preventing a cell proliferative disorder can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

Administering a composition of the present invention to a cell or a subject in need thereof can result in modulation (i.e., stimulation or inhibition) of an activity of a protein methyltransferase of interest.

Treating cancer or a cell proliferative disorder can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc. Natl. Acad. Sci. USA. 100(5): 2674-8, 2003. In an aspect, cell death occurs by apoptosis.

Preferably, an effective amount of a composition of the present invention, or a pharmaceutically acceptable salt or solvate thereof, is not significantly cytotoxic to normal cells. A therapeutically effective amount of a compound is not significantly cytotoxic to normal cells if administration of the compound in a therapeutically effective amount does not induce cell death in greater than 10% of normal cells. A therapeutically effective amount of a compound does not significantly affect the viability of normal cells if administration of the compound in a therapeutically effective amount does not induce cell death in greater than 10% of normal cells. In an aspect, cell death occurs by apoptosis.

Contacting a cell with a composition of the present invention, or a pharmaceutically acceptable salt or solvate thereof, can induce, or activate cell death selectively in cancer cells. Administering to a subject in need thereof a compound of the present invention, or a pharmaceutically acceptable salt or solvate thereof, can induce or activate cell death selectively in cancer cells. Contacting a cell with a composition of the present invention, or a pharmaceutically acceptable salt or solvate thereof, can induce cell death selectively in one or more cells affected by a cell proliferative disorder. Preferably, administering to a subject in need thereof a composition of the present invention, or a pharmaceutically acceptable salt or solvate thereof, induces cell death selectively in one or more cells affected by a cell proliferative disorder.

The present invention relates to a method of treating or preventing cancer by administering a composition of the present invention, or a pharmaceutically acceptable salt or solvate thereof, to a subject in need thereof, where administration of the composition of the present invention, or a pharmaceutically acceptable salt or solvate thereof, results in one or more of the following: prevention of cancer cell proliferation by accumulation of cells in one or more phases of the cell cycle (e.g. G1, G1/S, G2/M), or induction of cell senescence, or promotion of tumor cell differentiation; promotion of cell death in cancer cells via cytotoxicity, necrosis or apoptosis, without a significant amount of cell death in normal cells, antitumor activity in animals with a therapeutic index of at least 2. As used herein, “therapeutic index” is the maximum tolerated dose divided by the efficacious dose.

The term “kit” means a combination partners as defined above can be dosed independently or by use of different fixed combinations with distinguished amounts of the combination partners, i.e. simultaneously or at different time points. The parts of the kit of parts can then, e.g., be administered simultaneously or chronologically staggered, that is at different time points and with equal or different time intervals for any part of the kit of parts. The ratio of the total amounts of the combination partners to be administered in the combined preparation can be varied. The combination partners can be administered by the same route or by different routes.

One skilled in the art may refer to general reference texts for detailed descriptions of known techniques discussed herein or equivalent techniques. These texts can, of course, also be referred to in making or using an aspect of the invention.

### EXAMPLES

In order that the disclosure disclosed herein may be more efficiently understood, examples are provided below. It should be understood that these examples are for illustrative purposes only and are not to be construed as limiting the disclosure in any manner.

LP-100 (Irofulven) and LP-184 ((−)-hydroxyureamethyl acylfulvene) belong to the acylfulvene compound family known to induce DNA lesions repaired by the Transcription-Coupled Nucleotide Excision Repair (TC-NER) pathway. If the TC-NER pathway is impaired, DNA damage can no longer be repaired, and cell death will occur.

PARP inhibitor selectively induces cell death and inhibits the growth of cancer cells. In combination with an acylfulvene, the cells or tumors were killed or reduced.

### Example 1

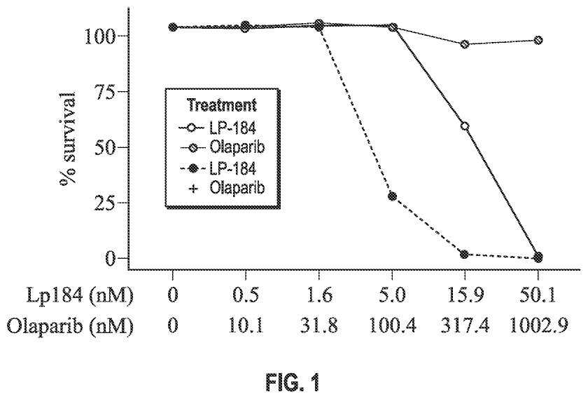

The effect of the combination of LP-184 with Olaparib on the survival of cells was effective. More particularly, the results are shown in FIG. 1. LP-184 and Olaparib combination resulted in reduced cell survival compared to LP-184 alone or Olaparib alone in the Ovarian cancer cell line OVCAR3.

### Example 2

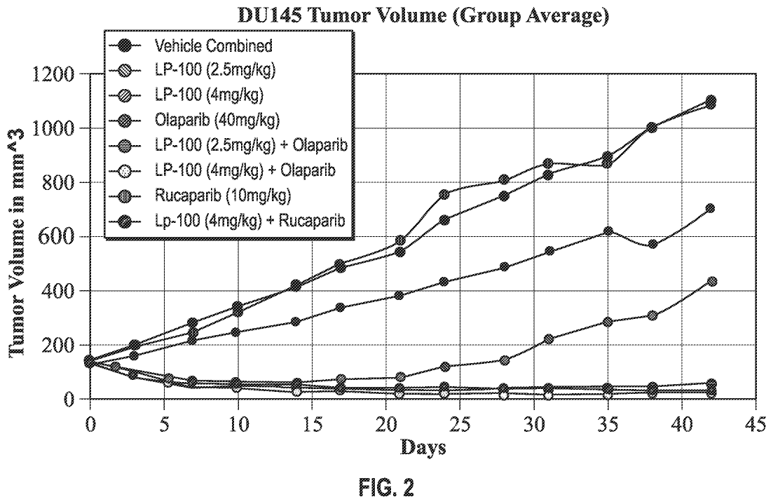

The effect of the combination of LP-100 with Olaparib on the survival of prostate cancer xenograft mouse model cells was effective. More particularly, the results are shown in FIG. 2. LP-100 and Olaparib combination resulted in reduced tumor shrinkage compared to LP-100 alone or Olaparib alone in the Prostate cancer xenograft mouse model DU145.

### Example 3

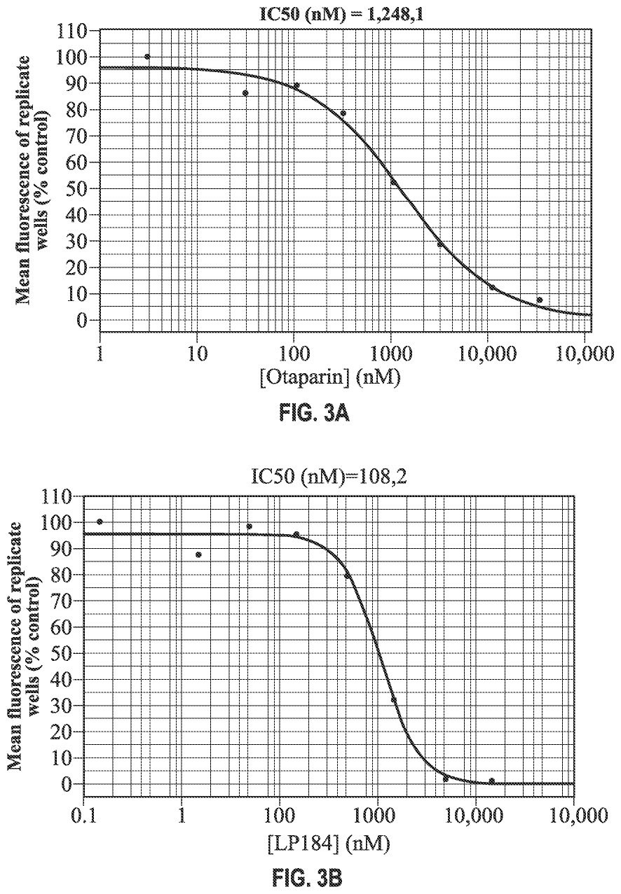

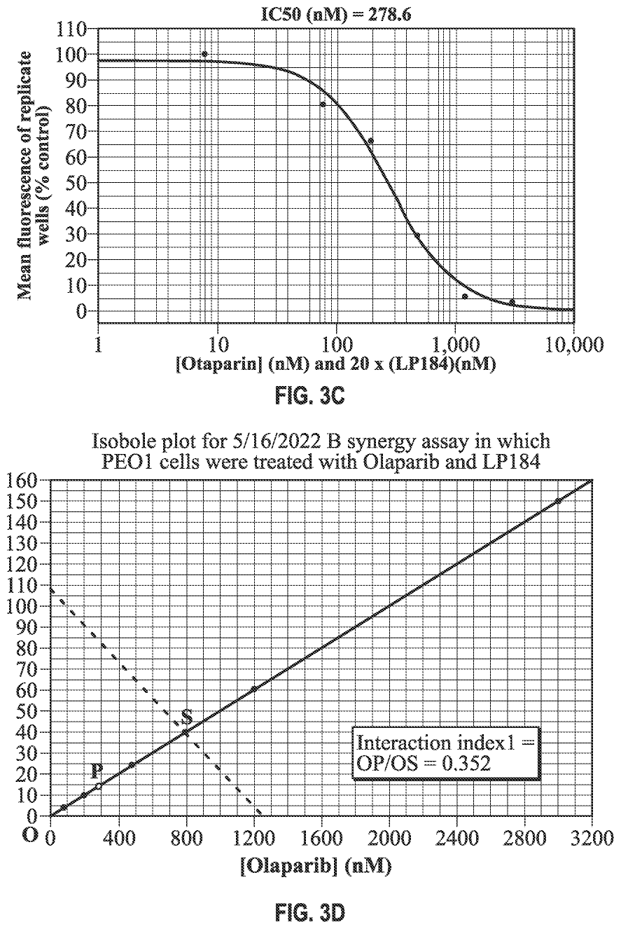

FIGS. 3A, 3B, 3C, and 3D show that LP-184 and Olaparib have a synergistic effect on the BRCA2 mutant ovarian tumor cell line PEO1. Cell sensitivity analysis was performed to evaluate the synergistic effect of LP-184 and Olaparib on PEO1 cell line, which is an established BRCA2 mutant ovarian tumor cell line. Cells were seeded onto 96-well plate and were continuously exposed to drug treatment for 8.5 days. IC50 data were obtained from Olaparib, LP-184 single treatment and combination treatment. IC50 data for Olaparib and LP184 alone was 1248.1 nM and 108.2 nM respectively (FIGS. 3A and 3B). In contrast, 50% of cell growth inhibition was obtained by using 278.6 nM Olaparib and 13.93 nM LP-184 together (FIG. 3C), which was 4.48-fold less and 7.77-fold less respectively compared to single drug treatment. Furthermore, as shown in FIG. 3D, the synergistic effect of Olaparib and LP-184 co-treatment was analyzed by using an Isobole plot. The interaction index was 0.352, indicating synergism (Tallarida, 2012).

IC50 data of Olaparib and LP-184 single treatment was 1248.1 nM (FIG. 3A) and 108.2 nM (FIG. 3B) respectively. IC50 data of Olaparib and LP-184 co-treatment was 278.6 nM Olaparib and 13.93 nM LP-184 (FIG. 3C). The synergistic effect of LP-184 and Olaparib was analyzed through an Isobole plot (FIG. 3Dt). The interaction index was 0.352.

### Example 4

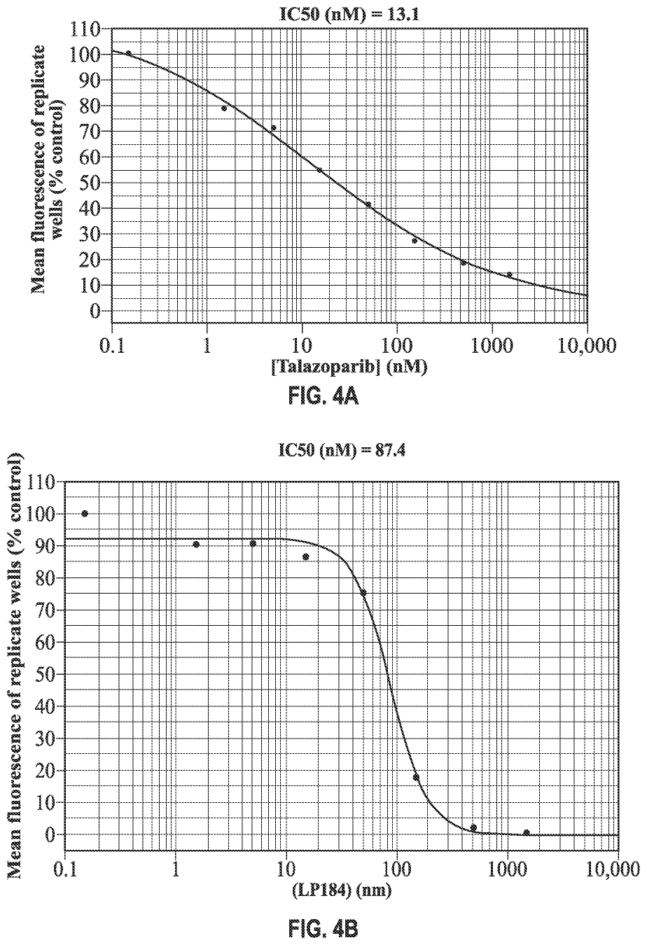

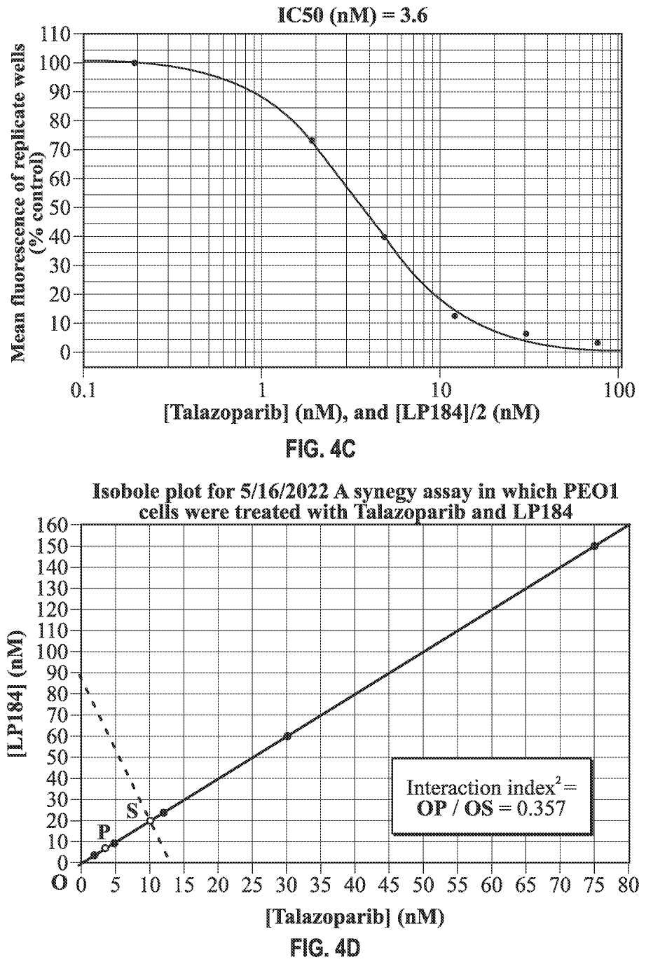

Cell sensitivity analysis was performed to evaluate the synergistic effect of LP-184 and Talazoparib on PEO1 cell line. Experiments were done as described above. IC50 data for Talazoparib and LP184 alone was 13.1 nM and 87.4 nM respectively (FIG. 4A and FIG. 4B). In contrast, cell growth was inhibited by 50% when combining 3.6 nM Talazoparib and 7.2 nM LP-184 together (FIG. 4C), which was 3.64-fold less and 12.14-fold less respectively compared to single drug treatment. Furthermore, the synergistic effect of Talazoparib and LP-184 co-treatment was analyzed by using an Isobole plot (FIG. 4D). The interaction index was 0.357, indicating synergism.

The effect of the combination of LP-184 with Talazoparib on the survival of cells was effective. FIGS. 4A-4D shows LP-184 and Talazoparib have synergistic effect on BRCA2 mutant ovarian cancer cell line PEO1. IC50 data of Talazoparib and LP-184 single treatment was 13.1 nM (FIG. 4A) and 87.4 nM (FIG. 4B) respectively. IC50 of Talazoparib and LP-184 co-treatment was 3.6 nM Olaparib and 7.2 nM LP-184 (FIG. 4C). Synergistic effect of LP-184 and Talazoparib was analyzed through an Isobole plot (FIG. 4D). The interaction index was 0.357.

### Example 4—Synergy Score

MacSynergy II software was used to score the combination of LP-184/LP-100 and PARP inhibitor. This program allows the three-dimensional examination of drug interactions of all data points generated from the checkerboard combination of two inhibitors with Bliss-Independence model (not shown). Confidence bounds are determined from replicate data. If the 95% confidence limits (CL) do not overlap the theoretic additive surface, then the interaction between the two drugs differs significantly from additive. The volumes of synergy or antagonism can be determined and graphically depicted in three dimensions and represent the relative quantity of synergism or antagonism per change in the two drug concentrations. Synergy and antagonism volumes are based on the Bliss independence model, which assumes that both compounds act independently on different targets. A set of predicted fractional responses faAB under the Bliss independence model is calculated as faAB−faA+faB−faA faB with faA and faB representing the fraction of possible responses, e.g. percent (%) inhibition, of compounds A and B at amounts dA and dB, respectively, and describes the percent inhibition of a combination of compounds A and B at amount (dA+dB). A bliss synergy score >10 indicates synergy between the two testing compounds. Table 1 shows the bliss synergistic score of LP-100 and Olaparib, Rucaparib, and Niraparib.

TABLE 1
PARP inhibitor synergizes with LP-100

| Cancer | | PARP | Bliss Synergy Score between |
|---|---|---|---|
| Type | Cell Line | inhibitor | LP-100 and PARP inhibitor |
| Prostate | 22RV1 | Rucaparib | 18.57 |
| Ovarian | OVCAR3 | Olaparib | 12.19 |
| Ovarian | OVCAR3 | Rucaparib | 14.11 |
| Ovarian | OVCAR3 | Niraparib | 10.34 |

While a number of exemplary aspects and embodiments have been discussed above, those of skill in the art will recognize certain modifications, permutations, additions, and sub-combinations thereof. It is therefore intended that the following appended claims and claims hereafter are interpreted to include all such modifications, permutations, additions, and sub-combinations as are within their true spirit and scope.

1. A method of treating cancer, the method comprises administering to a subject in need of treatment a combination of active agents comprising:
a. a therapeutically effective amount of an illudin or an illudin analog thereof, derivative, or a pharmaceutically acceptable salt thereof; and
b. a therapeutically effective amount of a PARP inhibitor or an analog, derivative, or a pharmaceutically acceptable salt thereof.

2. The method of claim 1, wherein the illudin analog is an acylfulvene.

3. The method of claim 2, wherein the acylfulvene is HydroxyUreaMethylAcylfulvene.

4. The method of claim 1, wherein the illudin analog has the following structure:

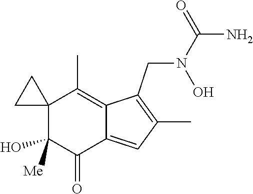 [SMILES: CC1=C(CN(O)C(N)=O)C2=C(C)C3(CC3)[C@@](C)(O)C(=O)C2=C1]

5. The method of claim 1, wherein the illudin analog has the following structure:

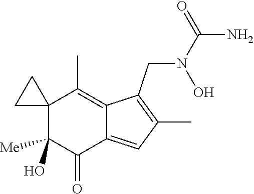 [SMILES: CC1=C(CN(O)C(N)=O)C2=C(C)C3(CC3)[C@](C)(O)C(=O)C2=C1]

6. The method of claim 1, wherein the PARP inhibitor is Talazoparib.

7. The method of claim 1, wherein the PARP inhibitor is selected from the group consisting of rucaparib, olaparib, veliparib, iniparib, niraparib, talazoparib, and a mixture thereof.

8. The method of claim 1, wherein the PARP inhibitor is olaparib.

9. The method of claim 1, wherein the PARP inhibitor is administrated at a dose of 20 mg, 40 mg or 60 mg twice daily.

10. The method of claim 1, wherein the active agents are administered separately.

11. The method of claim 1, wherein the active agents are administered daily.

12. The method of claim 1, wherein the active agents are administered sequentially.

13. The method of claim 1, wherein the active agents are administered as a co-formulation.

14. The method of claim 1, wherein an illudin or an analog thereof administration is before, during, or after PARP inhibitor administration.

15. The method of claim 1, the method further comprising administering radiotherapy, chemotherapy to, performing surgery on, the subject before, during, or following the illudin and/or administering the PARP inhibitor.

16. The method of claim 1, wherein the cancer is colorectal cancer, pancreatic cancer, primary liver cancers, kidney cancer, ovarian cancer, uterine cancer, lung cancer, breast cancer, prostate cancer, sarcomas, or adipose tissue cancer

17. The method of claim 1, wherein the subject is an animal.

18. The method of claim 1, wherein the subject or mammal is a human.

19. The method of claim 2 or 3, further comprising subjecting the subject to radiation therapy before, after, or during treatment with HydroxyUreaMethyl Acylfulvene.

20. The method of claim 1, further comprising administering an additional therapeutic agent selected from the group consisting of cisplatin, paclitaxel, and other available therapies.

21. The method of claim 1, wherein the cancer comprises a solid tumor.

22. The method of claim 18, wherein the solid tumor is a tumor of the breast, central nervous system, colon, skin, lung, ovary, prostate, pancreatic or kidney.

23. The method of claim 1, wherein the cancer is lymphoma, leukemia, or melanoma.

24. The method of claim 1, wherein the PARP inhibitor is administrated orally at a dose of 1-120 mg twice daily.

25. A pharmaceutical composition comprising a therapeutically effective amount of an illudin or an illudin analog thereof, derivative, or a pharmaceutically acceptable salt thereof; and a therapeutically effective amount of a PARP inhibitor or an analog, derivative, or a pharmaceutically acceptable salt thereof.

26. The pharmaceutical composition claim 25, wherein the illudin analog is HydroxyUreaMethylAcylfulvene.

27. The pharmaceutical composition of claim 25, wherein the illudin analog has the following structure:

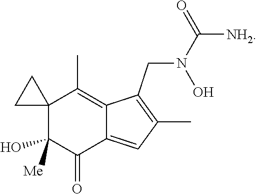 [SMILES: CC1=C(CN(O)C(N)=O)C2=C(C)C3(CC3)[C@@](C)(O)C(=O)C2=C1]

28. The pharmaceutical composition of claim 25, wherein the illudin analog has the following structure:

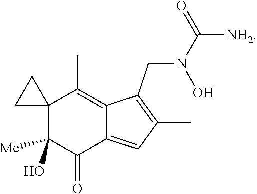 [SMILES: CC1=C(CN(O)C(N)=O)C2=C(C)C3(CC3)[C@](C)(O)C(=O)C2=C1]

29. The pharmaceutical composition of claim 25, wherein the PARP inhibitor is olaparib.

30. A kit for the treatment of cancer in a subject comprising a therapeutically effective amount of HydroxyUreaMethylAcylfulvene, or a pharmaceutically acceptable salt thereof; and a therapeutically effective amount of a PARP inhibitor or an analog, derivative, or a pharmaceutically acceptable salt thereof.

31. The pharmaceutical composition claim 30, wherein the PARP inhibitor is selected from the group consisting of rucaparib, olaparib, veliparib, iniparib, niraparib, talazoparib, and a mixture thereof.

32. The pharmaceutical composition of claim 30, wherein the illudin analog has the following structure:

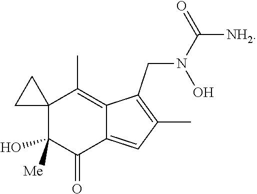 [SMILES: CC1=C(CN(O)C(N)=O)C2=C(C)C3(CC3)[C@@](C)(O)C(=O)C2=C1]

33. The pharmaceutical composition of claim 30, wherein the PARP inhibitor is olaparib.

34. The pharmaceutical composition of claim 30, wherein the illudin analog has the following structure: 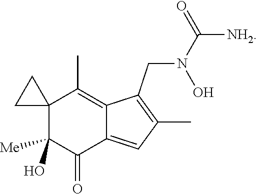 [SMILES: CC1=C(CN(O)C(N)=O)C2=C(C)C3(CC3)[C@](C)(O)C(=O)C2=C1]
